SEQUENCE LISTING (2 sequences)

SEQ ID NO: 1 - DNA, 1041 bp, synthetic construct
  source 1..1041: mol_type = other DNA
atgcgtgctctgcagtacaccgaaatcggttctgaaccggttgttgttgacctgccgaccccggctccgggtccgggtgaaatcctgctgaaagttaccgctgctggtctgtgccactctgacatcttcgttatggacatgccggctgaacagtacgcttacggtctgccgctgaccctgggtcacgaaggtgttggtaccgttgctgaactgggtgacggtgttaccggtttcgaaaccggtgacgctgttgctgtttacggtccgtggggttgcggtgcttgccacgcttgcgctcgtggtcgtgaaaactactgcacccgtgctgctgaactgggtatcaccccgccgggtctgggttctccgggttctatggctgaatacatgatcgttgactctgctcgtcacctggttccgatcggtgacctggacccggttgctgctgttccgctgaccgacgctggtctgaccccgtaccacgctatctctcgtgttctgccgctgctgggtccgggttctaccgctgttgttatcggtgttggtggtctgggtcacgttggtatccagatcctgcgtgctgtttctgctgctcgtgttatcgctgttgacctggacgacgaccgtctggctctggctcgtgaagttggtgctgacgctgctgttaaatctggtgctggtgctgctgacgctatccgtgaactgaccggtggtgaaggtgctaccgttgttttcgacttcgttggtgctcagtctaccatcgacatggctcagcaggttgttgctatcgacggtcacatctctatcgttggtatccacgctggtgctcacgctaaagttggtttcttcatgatcccgttcggtgcttctgttgttaccccgtactggggtacccgttctgaactgatggaagttgttgacctggctcgtgctggtcgtctggacatccacaccgaaaccttcaccctggacgaaggtccgaccgcttaccgtcgtctgcgtgaaggttctatccgtggtcgtggtgttgttgttccgggttaa

SEQ ID NO: 2 - protein, 346 aa, synthetic construct
MRALQYTEIGSEPVVVDLPTPAPGPGEILLKVTAAGLCHSDIFVMDMPAEQYAYGLPLTLGHEGVGTVAELGDGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDLDDDRLALAREVGADAAVKSGAGAADAIRELTGGEGATVVFDFVGAQSTIDMAQQVVAIDGHISIVGIHAGAHAKVGFFMIPFGASVVTPYWGTRSELMEVVDLARAGRLDIHTETFTLDEGPTAYRRLREGSIRGRGVVVPG

(19) United States
(12) Patent Application Publication
(10) Pub. No.: US 2026/0337647 A1
(43) Pub. Date: Oct. 29, 2026

(54) ENZYME-CATALYZED PREPARATION METHOD FOR (S)-1-BOC-3-HYDROXYPIPERIDINE

(71) Applicant: Nanjing Normal University Changzhou Institute of Synthetic Biology Industry Co., Ltd., Changzhou (CN)

(72) Inventors: Zhihua JIN, Changzhou (CN); Yudi BAO, Changzhou (CN); Bingsheng WANG, Changzhou (CN); Wenjun FENG, Changzhou (CN); Xueyan LIN, Changzhou (CN); Jingle LIANG, Changzhou (CN); Wenhe SHEN, Changzhou (CN); Qing XU, Changzhou (CN)

(21) Appl. No.: 19/697,655

(22) Filed: Jun. 4, 2026

(30) Foreign Application Priority Data
Jan. 8, 2026 (CN) 202610021317.6

(51) Int. Cl. C12P 17/12

(52) U.S. Cl. CPC C12P 17/12

(57) ABSTRACT

An enzyme-catalyzed preparation method for (S)-1-Boc-3-hydroxypiperidine, this method belongs to the technical field of biopharmaceuticals, especially relating to the enzyme-catalyzed preparation method for (S)-1-Boc-3-hydroxypiperidine, comprising using Alcohol Dehydrogenase (ADH) as a catalyst to catalyze the reduction reaction of N-Boc-3-piperidone to obtain(S)-1-Boc-3-hydroxypiperidine, the amino acid sequence of the ADH is shown in SEQ ID NO.2. The enzyme-catalyzed preparation method provided by the present disclosure uses inexpensive Nicotinamide Adenine Dinucleotide (NAD) as a coenzyme, in combination with a highly catalytically active ADH that also possesses coenzyme regeneration capability, thereby achieving a method for preparing (S)-1-Boc-3-hydroxypiperidine using only a specific ADH and an inexpensive NAD coenzyme. Meanwhile, the acceptable substrate concentration during the catalysis process is increased, and the production cost of (S)-1-Boc-3-hydroxypiperidine is reduced.

### SEQUENCE LISTING

The XML file named “Sequence_Listing.xml,” created on Jun. 4, 2026, and having a size of 4,456 bytes, contains the sequence listing for this application and is hereby incorporated by reference in its entirety.

### TECHNICAL FIELD

The present disclosure belongs to the technical field of biopharmaceuticals, especially relating to an enzyme-catalyzed preparation method for (S)-1-Boc-3-hydroxypiperidine.

### BACKGROUND

(S)-1-tert-Butoxycarbonyl-3-hydroxypiperidine ((S)-1-Boc-3-hydroxypiperidine) is an important chiral building block widely used in medicinal chemistry and fine chemicals. It serves as a key chiral intermediate, particularly in the synthesis of various bioactive molecules such as protease inhibitors and neurotransmitter modulators. Its optical purity directly affects the efficacy and safety of the final drug. Therefore, developing efficient, highly selective methods for producing the(S)-configured product with high optical purity holds significant industrial value.

Currently, methods for preparing chiral 3-hydroxypiperidine derivatives mainly include chemical asymmetric synthesis, such as asymmetric reduction induced by chiral auxiliaries or catalytic hydrogenation using chiral catalysts, which often requires expensive chiral ligands or catalysts and harsh reaction conditions like high-pressure hydrogen, while the enantioselectivity can sometimes fall short of extremely high standards, and the subsequent removal of chiral auxiliaries during work-up adds to process complexity and cost; traditional biocatalysis employs whole microbial cells or crude enzyme preparations for the asymmetric reduction of ketones. Although this approach offers good stereoselectivity, it generally suffers from several limitations. The intracellular multi-enzyme system may lead to side reactions, reducing product purity and yield. A complex coenzyme regeneration system is often required, making process development cumbersome. Substrate concentrations are typically keep low, often below 50 g/L, to avoid inhibition of the biocatalyst by either the substrate or the product. This results in low productivity, necessitates large equipment volumes, and incurs high downstream separation costs; and multi-enzyme cascade reactions, a recently developed synergistic catalytic system that achieves one-pot synthesis of the target chiral alcohol from prochiral ketones by coupling ketoreductases (KRED), coenzyme-regenerating enzymes, and others, yet this approach requires precise matching of reaction conditions, expression levels, and stability across multiple enzymes, making process development and optimization difficult, while the use of multiple enzymes increases production costs and introduces uncertainty in process control.

Among currently reported methods, the number of Alcohol Dehydrogenase (ADH) or KRED capable of achieving efficient and highly selective reduction is limited. Moreover, most of these enzymes suffer from severe activity inhibition or loss of selectivity when confronted with the high substrate concentrations required for industrial application, which restricts their large-scale use.

### SUMMARY

To solve above technical problems, the present disclosure proposes an enzyme-catalyzed preparation method for (S)-1-Boc-3-hydroxypiperidine, the enzyme-catalyzed preparation method provided by the present disclosure uses inexpensive Nicotinamide Adenine Dinucleotide (NAD) as a coenzyme, in combination with a highly catalytically active Alcohol Dehydrogenase (ADH) that also possesses coenzyme regeneration capability, thereby achieving a method for preparing (S)-1-Boc-3-hydroxypiperidine using only a specific ADH and the inexpensive NAD coenzyme. Meanwhile, an acceptable substrate concentration during a catalysis process is increased, and a production cost of (S)-1-Boc-3-hydroxypiperidine is reduced.

To achieve the above purposes, the present disclosure provides an enzyme-catalyzed preparation method for (S)-1-Boc-3-hydroxypiperidine, including using an ADH as a catalyst to catalyze a reduction of N-Boc-3-piperidone to obtain (S)-1-Boc-3-hydroxypiperidine; an amino acid sequence of the ADH is shown in SEQ ID NO.2.

In some embodiments, a concentration of ADH is 1-15 g/L.

In some embodiments, the ADH exists in one or several forms of enzyme powder, cells expressing ADH, disrupted cell lysate of cells expressing ADH, immobilized ADH, or immobilized cells expressing ADH.

In some embodiments, the reduction reaction is carried out at pH 6-8 and a temperature of 25-45 °C.

In some embodiments, a concentration of N-Boc-3-piperidone is 100-300 g/L.

In some embodiments, the reduction reaction uses NAD as a coenzyme.

Further, a concentration of NAD is 0.01-1 g/L.

In some embodiments, the reduction reaction is carried out in a buffer solution of phosphate buffer or triethanolamine buffer.

Further, a concentration of the buffer is 50-100 mmol/L.

In some embodiments, this method further includes following steps: Filtering a mixture after the reduction reaction is completed, extracting a filtrate with an organic solvent, then concentrating and crystallizing to obtain purified (S)-1-Boc-3-hydroxypiperidine.

Compared with the existing technologies, the present disclosure has following advantages and technical effects:

The present disclosure provides an enzyme-catalyzed preparation method for (S)-1-Boc-3-hydroxypiperidine, a reduction reaction process that only requires a specific ADH, the specific ADH is derived from Rhodococcus pyridinivorans, with the amino acid sequence as shown in SEQ ID NO.2, accompanied by inexpensive NAD as a coenzyme, N-Boc-3-piperidone reduced may be to prepare (S)-1-Boc-3-hydroxypiperidine. A purity of (S)-1-Boc-3-hydroxypiperidine prepared by the enzyme-catalyzed preparation method of the present disclosure is greater than 99.5%, and an optical purity is greater than 99.9%, an acceptable maximum substrate concentration is increased to 300 g/L, which reduces the production cost of (S)-1-Boc-3-hydroxypiperidine and provides significant industrial application value.

### BRIEF DESCRIPTION OF THE DRAWINGS

To more clearly illustrate the technical solutions in the embodiments of the present disclosure or in the prior art, the following briefly introduces the drawings required for the embodiments. Obviously, the drawings described below are merely some embodiments of the present disclosure, and for those of ordinary skill in the art, other drawings may be obtained based on these drawings without any creative effort.

FIG. 1 is a high performance liquid chromatogram of the process reaction system during the reduction reaction process of Embodiment 1.

### DETAILED DESCRIPTION

Hereinafter, various exemplary embodiments of the present disclosure are described in detail. This detailed description should not be construed as limiting the present disclosure, but rather as providing a more detailed explanation of certain aspects, features, and embodiments of the disclosure. It should be understood that the terms used herein are merely for describing particular embodiments and are not intended to limit the present disclosure. Furthermore, for any numerical range outlined in the present disclosure, it should be understood that each intermediate value between the upper and lower limits of that range is also specifically disclosed. Each smaller range between any stated value or intermediate value within a stated range and any other stated value or intermediate value within that range is also encompassed within the present disclosure. The upper and lower limits of these smaller ranges may be independently included or excluded from the range. Unless otherwise specified, all technical and scientific terms used herein have the same meanings as commonly understood by those of ordinary skill in the art to which the present disclosure belongs. Although only preferred methods and materials are described herein, any methods and materials similar or equivalent to those described herein may also be used in the practice or testing of the present disclosure. All references mentioned in this specification are incorporated by reference to disclose and describe the methods and/or materials associated with those references. In the event of conflict with any incorporated reference, the content of this specification shall control. Various modifications and changes may be made to the specific embodiments described in this specification without departing from the scope or spirit of the present disclosure, as will be apparent to those skilled in the art. Other embodiments derived from the description of the present disclosure will be apparent to those skilled in the art. The description and examples herein are merely illustrative. As used herein, terms such as “comprise,” “include,” “have,” “contain,” and the like are open-ended language, meaning including but not limited to.

The source of the material used in the present disclosure: The Alcohol Dehydrogenase (ADH) used in the present disclosure is derived from Rhodococcus pyridinivorans, NCBI accession number is WP_024100401.1; the pET-28a vector is purchased from Sangon Biotech (Shanghai) Co., Ltd.; Escherichia coli (E. coli) BL21 (DE3) is purchased from Sangon Biotech (Shanghai) Co., Ltd.; the Lysogeny Broth (LB) medium is purchased from Sangon Biotech (Shanghai) Co., Ltd.

The formula of phosphate buffer used in the present disclosure is as follows: 1. 361 g of KH₂PO₄ (corresponding to 0. 01 mol) is weighed in a beaker and dissolved in 80 mL of water; monitored by a pH meter, 0.1 M NaOH solution is used to slowly titrate while stirring until the pH is stable at 7.0; the solution is transferred to a volumetric flask and diluted to 100 mL with water to obtain 100 mM buffer.

The preparation method of ADH used in this present disclosure: According to the amino acid sequence (SEQ ID NO.2) of the target ADH, the whole gene synthesis is performed for the preferred codons of E. coli (the nucleotide sequence encoding the ADH is shown in SEQ ID NO.1), and the Nde I and Nhe I restriction enzyme digestion sites matching the pET-28a vector are introduced at the 5′ and 3′ ends, respectively, and inserted into the pET28a plasmid to obtain the pET28a-ADH recombinant plasmid. The pET28a-ADH recombinant plasmid is transformed into the expression host E. coli BL21 (DE3). The specific operation is as follows: 50 ng of pET28a-ADH recombinant plasmid is added to 100 μL of E. coli BL21 (DE3) competent cells, gently mixed by pipetting, and placed in an ice-water bath for 30 min. The cells are heat shocked at 42 °C for 90 s and incubated in ice water for 8 min. Then, 500 μL of LB medium without antibiotics is added and incubated at 37 °C for 60 min to allow recovery growth and expression of the antibiotic resistance gene. Subsequently, 50 μL of bacterial solution is evenly coated on LB plates containing 50 mg/L kanamycin. The plate is placed upright until the liquid is absorbed, then inverted and incubated in a constant-temperature incubator at 37 °C for 14 h. The positive clones of colony Polymerase Chain Reaction (PCR) are selected, purified by streaking, cultured in a shaker, and the plasmids are extracted, followed by restriction enzyme digestion verification and sequencing verification. Finally, the correctly verified positive clones are obtained, and the recombinant E. coli engineering strain E. coli BL21 (DE3) (pET28a-ADH) of the target ADH is obtained for subsequent enzyme expression and enzyme-catalytic reaction.

The constructed engineered E. coli strain producing ADH is coated on an LB plate containing 50 mg/L of kanamycin and cultured overnight at 37 °C. After that, a single colony is selected and inoculated into 50 mL of liquid LB medium containing 50 mg/L of kanamycin and cultured at 37 °C. After 12 h of culture, 2.5 mL of seed solution is transferred into 50 mL of fresh LB liquid medium containing 50 mg/L of kanamycin and cultured at 37 °C. When the Optical Density at 600 nm (OD₆₀₀) reaches 0.6-0.8, the temperature is reduced to 28 °C, and Isopropyl β-D-1-Thiogalactopyranoside (IPTG) with a final concentration of 0.1 mM is added for induction and culture for 14 h. The cells are collected by centrifugation at 5000 rpm to obtain the wet cells of the recombinant strain expressing the ADH. The wet cells are resuspended in phosphate buffer (pH 7.0) to a concentration of 10-20%, ultrasonically broken in an ice bath, and the supernatant is collected by centrifugation to obtain the crude enzyme solution of the recombinant expression strain.

The nucleotide sequence encoding ADH is shown in SEQ ID NO.1, SEQ ID NO.1: ATGCGTGCTCTGCAGTACACCGAAATCGGTTCTGAACCGGTTGTTGTTG ACCTGCCGACCCCGGCTCCGGGTCCGGGTGAAATCCTGCTGAAAGTTACCGC TGCTGGTCTGTGCCACTCTGACATCTTCGTTATGGACATGCCGGCTGAACAGT ACGCTTACGGTCTGCCGCTGACCCTGGGTCACGAAGGTGTTGGTACCGTTGC TGAACTGGGTGACGGTGTTACCGGTTTCGAAACCGGTGACGCTGTTGCTGTT TACGGTCCGTGGGGTTGCGGTGCTTGCCACGCTTGCGCTCGTGGTCGTGAAA ACTACTGCACCCGTGCTGCTGAACTGGGTATCACCCCGCCGGGTCTGGGTTC TCCGGGTTCTATGGCTGAATACATGATCGTTGACTCTGCTCGTCACCTGGTTC CGATCGGTGACCTGGACCCGGTTGCTGCTGTTCCGCTGACCGACGCTGGTCT GACCCCGTACCACGCTATCTCTCGTGTTCTGCCGCTGCTGGGTCCGGGTTCTA CCGCTGTTGTTATCGGTGTTGGTGGTCTGGGTCACGTTGGTATCCAGATCCTG CGTGCTGTTTCTGCTGCTCGTGTTATCGCTGTTGACCTGGACGACGACCGTCT GGCTCTGGCTCGTGAAGTTGGTGCTGACGCTGCTGTTAAATCTGGTGCTGGT GCTGCTGACGCTATCCGTGAACTGACCGGTGGTGAAGGTGCTACCGTTGTTT TCGACTTCGTTGGTGCTCAGTCTACCATCGACATGGCTCAGCAGGTTGTTGCT ATCGACGGTCACATCTCTATCGTTGGTATCCACGCTGGTGCTCACGCTAAAGT TGGTTTCTTCATGATCCCGTTCGGTGCTTCTGTTGTTACCCCGTACTGGGGTAC CCGTTCTGAACTGATGGAAGTTGTTGACCTGGCTCGTGCTGGTCGTCTGGAC ATCCACACCGAAACCTTCACCCTGGACGAAGGTCCGACCGCTTACCGTCGTC TGCGTGAAGGTTCTATCCGTGGTCGTGGTGTTGTTGTTCCGGGTTAA.

The amino acid sequence of ADH is shown in SEQ ID NO.2, SEQ ID NO.2: MRALQYTEIGSEPVVVDLPTPAPGPGEILLKVTAAGLCHSDIFVMDMPAEQYAY GLPLTLGHEGVGTVAELGDGVTGFETGDAVAVYGPWGCGACHACARGRENYC TRAAELGITPPGLGSPGSMAEYMIVDSARHLVPIGDLDPVAAVPLTDAGLTPYHA ISRVLPLLGPGSTAVVIGVGGLGHVGIQILRAVSAARVIAVDLDDDRLALAREVG ADAAVKSGAGAADAIRELTGGEGATVVFDFVGAQSTIDMAQQVVAIDGHISIVG IHAGAHAKVGFFMIPFGASVVTPYWGTRSELMEVVDLARAGRLDIHTETFTLD EGPTAYRRLREGSIRGRGVVVPG.

### Embodiment 1

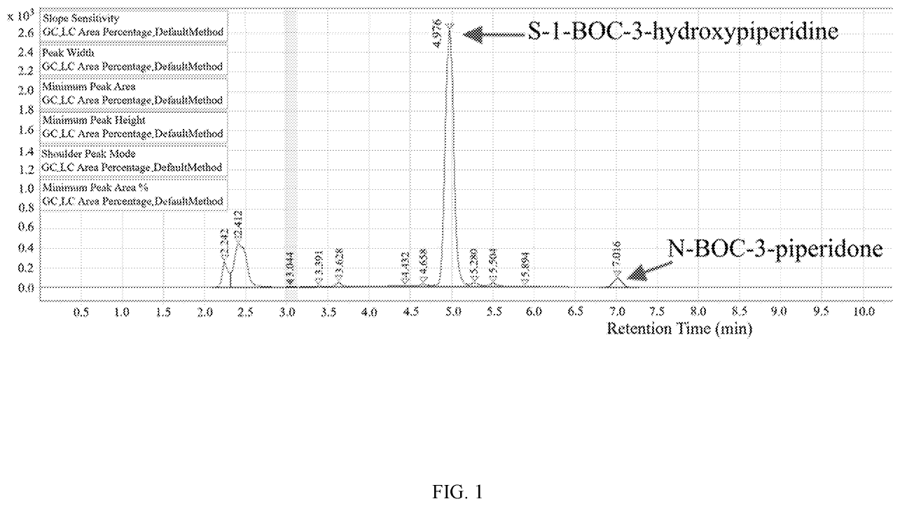

0.4 L of pre-prepared 100 mM phosphate buffer with pH 7.0, 3 g of wet cell of recombinant strain expressing ADH, and 0.03 g of Nicotinamide Adenine Dinucleotide (NAD) are added to a 1 L round-bottom flask. After being mixed uniformly, 60 g of N-Boc-3-piperidone and 0.1 L of isopropanol are added, followed by the addition of 100 mM phosphate buffer with pH 7.0 to bring the reaction system to a total volume of 0.6 L. The reduction reaction is carried out with stirring at 37 °C for 12 h. The reaction mixture is then filtered by vacuum filtration through a filter membrane at −0.095 MPa, the filtrate is extracted with ethyl acetate, and the upper layer as the ethyl acetate phase is collected. The extract is concentrated under reduced pressure at −0.095 MPa and 40 °C to obtain 67 g of a pale yellow liquid as a crude product; the crude product is added to 0.4 kg of n-hexane, and the mixture is stirred at a temperature of 0-10 °C to precipitate a white solid, the solid is then collected by vacuum filtration through a filter membrane at −0.095 MPa and dried under vacuum at −0.095 MPa and 40 °C for 2 h to yield 56 g of (S)-1-Boc-3-hydroxypiperidine with a purity of 99.9%, an optical purity of 99.9%, and a yield of 93.3%. As shown in FIG. 1, the high performance liquid chromatogram of the reaction system during the reduction reaction process is presented.

Method for the determination and calculation of the purity and optical purity of the product(S)-1-Boc-3-hydroxypiperidine: The gas chromatograph 7820A is used, and the chromatographic column is HYDRODEXβ-TBDAC capillary column (25 m×0.25 mm; anpel, chiral column). The program is as follows: The detector is Flame Ionization Detector (FID) at 220 °C, the vaporization chamber temperature at 220 °C, the column temperature is maintained at 130 °C for 20 min, then increases to 150 °C at a rate of 10 °C/min and holds for 8 min, the column head pressure is 0.03 MPa, the hydrogen pressure is 0.05 MPa, the air pressure is 0.1 MPa, and the makeup gas pressure is 0.08 MPa.

The enantiomeric excess value of the product(S)-1-Boc-3-hydroxypiperidine is the optical purity (e.e. %) calculated by the following formula: Enantiomeric excess value=(S−R)/(S+R)×100%, where S is the concentration of is the concentration(S)-1-Boc-3-hydroxypiperidine and R of (R)-1-Boc-3-hydroxypiperidine.

The retention time of the substrate N-Boc-3-piperidone is 26.4 min, the retention time of (S)-1-Boc-3-hydroxypiperidine is 27.3 min, and the retention time of (R)-1-Boc-3-hydroxypiperidine is 27.7 min.

Formula for the calculation of yield: Yield (%)=(mass of product/mass of substrate)×100%.

### Embodiment 2

0.4 L of pre-prepared 100 mM phosphate buffer with pH 7.0, 6 g of wet cell of recombinant strain expressing ADH, and 0.06 g of NAD are added to a 1 L round-bottom flask. After being mixed uniformly, 120 g of N-Boc-3-piperidone and 0.1 L of isopropanol are added, followed by the addition of 100 mM phosphate buffer with pH 7.0 to bring the reaction system to a total volume of 0.6 L. The reduction reaction is carried out with stirring at 37 °C for 12 h. The reaction mixture is then filtered by vacuum filtration through a filter membrane at −0.095 MPa, the filtrate is extracted with ethyl acetate, and the upper layer as the ethyl acetate phase is collected. The extract is concentrated under reduced pressure at −0.095 MPa and 40 °C to obtain 133 g of a pale yellow liquid as a crude product; the crude product is added to 0.7 kg of n-hexane, and the mixture is stirred at a temperature of 0-10 °C to precipitate a white solid, the solid is then collected by vacuum filtration through a filter membrane at −0.095 MPa and dried under vacuum at −0.095 MPa and 40 °C for 2 h to yield 110 g of (S)-1-Boc-3-hydroxypiperidine with a purity of 99.5%, an optical purity of 99.9%, and a yield of 91.7%.

### Embodiment 3

0.4 L of pre-prepared 100 mM phosphate buffer with pH 7.0, 9 g of wet cell of recombinant strain expressing ADH, and 0.09 g of NAD are added to a 1 L round-bottom flask. After being mixed uniformly, 180 g of N-Boc-3-piperidone and 0.1 L of isopropanol are added, followed by the addition of 100 mM phosphate buffer with pH 7.0 to bring the reaction system to a total volume of 0.6 L. The reduction reaction is carried out with stirring at 37 °C for 12 h. The reaction mixture is then filtered by vacuum filtration through a filter membrane at −0.095 MPa, the filtrate is extracted with ethyl acetate, and the upper layer as the ethyl acetate phase is collected. The extract is concentrated under reduced pressure at −0.095 MPa and 40 °C to obtain 189 g of a pale yellow liquid as a crude product; the crude product is added to 1 kg of n-hexane, and the mixture is stirred at a temperature of 0-10 °C to precipitate a white solid, the solid is then collected by vacuum filtration through a filter membrane at −0.095 MPa and dried under vacuum at −0.095 MPa and 40 °C for 2 h to yield 156 g of (S)-1-Boc-3-hydroxypiperidine with a purity of 97.8%, an optical purity of 99.9%, and a yield of 86.7%.

The above-described embodiments are merely descriptions of preferred modes of the present disclosure and are not intended to limit the scope of the present disclosure. Without departing from the spirit of the present disclosure, any variations and improvements made to the technical solutions of the present disclosure by those of ordinary skill in the art shall fall within the protection scope determined by the claims of the present disclosure.

What is claimed is:

1. An enzyme-catalyzed preparation method for (S)-1-Boc-3-hydroxypiperidine, comprising using an Alcohol Dehydrogenase (ADH) as a catalyst to catalyze a reduction of N-Boc-3-piperidone to obtain(S)-1-Boc-3-hydroxypiperidine;
an amino acid sequence of the ADH is shown in SEQ ID NO.2.

2. The enzyme-catalyzed preparation method according to claim 1, wherein a concentration of ADH is 1-15 g/L.

3. The enzyme-catalyzed preparation method according to claim 1, wherein the ADH exists in one or several forms of enzyme powder, cells expressing ADH, disrupted cell lysate of cells expressing ADH, immobilized ADH, or immobilized cells expressing ADH.

4. The enzyme-catalyzed preparation method according to claim 1, wherein the reduction reaction is carried out at pH 6-8 and a temperature of 25-45 °C.

5. The enzyme-catalyzed preparation method according to claim 1, wherein a concentration of N-Boc-3-piperidone is 100-300 g/L.

6. The enzyme-catalyzed preparation method according to claim 1, wherein the reduction reaction uses NAD as a coenzyme.

7. The enzyme-catalyzed preparation method according to claim 6, wherein a concentration of the NAD is 0.01-1 g/L.

8. The enzyme-catalyzed preparation method according to claim 1, wherein the reduction reaction is carried out in a buffer solution of phosphate buffer or triethanolamine buffer.

9. The enzyme-catalyzed preparation method according to claim 8, wherein a concentration of the buffer is 50-100 mmol/L.

10. The enzyme-catalyzed preparation method according to claim 1, wherein this method further comprises following steps: filtering a mixture after the reduction reaction is completed, extracting a filtrate with an organic solvent, then concentrating and crystallizing to obtain purified(S)-1-Boc-3-hydroxypiperidine.
